(19) United States
(12) Patent Application Publication
(10) Pub. No.: US 2004/0202634 A1
(43) Pub. Date: Oct. 14, 2004

(54) Composition useful for cosmetic or dermatological use containing a block copolymer

(75) Inventor: Florence L'Alloret, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/785,955

(22) Filed: Feb. 26, 2004

Related U.S. Application Data
(60) Provisional application No. 60/459,643, filed on Apr. 3, 2003.

(30) Foreign Application Priority Data
Mar. 19, 2003 (FR) 0303356

(51) Int. Cl. A61K 7/06; A61K 7/11

(52) U.S. Cl. 424/70.16

(57) ABSTRACT

Composition containing at least one aqueous phase including at least one water-soluble or water-dispersible polymer, chosen from triblock or diblock polymers having a hydrophobic block with a glass transition temperature greater than or equal to 70° C. The composition has the advantage of being stable, even when it is in the form of an emulsion containing solid particles such as titanium dioxide. The invention also relates to the use of the composition, especially in cosmetics for caring for, cleansing, protecting and/or making up the skin, keratin fibers and/or mucous membranes.

### REFERENCE TO PRIOR APPLICATIONS

This application claims priority to U.S. provisional application No. 60/459,643 filed Apr. 3, 2003, and to French patent application 0303356 filed Mar. 19, 2003, both incorporated herein by reference.

### FIELD OF THE INVENTION

The present patent application relates to a cosmetic and/or dermatological composition comprising at least one aqueous phase including at least one water-soluble or water-dispersible polymer, chosen from triblock or diblock polymers comprising a hydrophobic block with a glass transition temperature greater than or equal to 70° C., and to uses thereof in cosmetics or dermatology, especially for caring for, cleansing, protecting and/or making up keratin materials (skin, mucous membranes or keratin fibres such as the hair and the eyelashes).

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

A large majority of cosmetic compositions comprise thickeners or gelling agents used to adjust the texture properties according to the intended application. The galenical supports that are the most representative are:

aqueous lotions or sera comprising little or no fatty phase,

emulsions, which are dispersions of two immiscible liquids such as water and oil. Oil-in-water (O/W) direct emulsions comprising an oily phase dispersed in an aqueous phase, are distinguished from water-in-oil (W/O) inverse emulsions, which are dispersions of an aqueous phase in an oily phase. There exist also multiple emulsions obtained after dispersing an inverse emulsion in an aqueous phase (W/O/W) or after dispersing a direct emulsion in an oily phase (O/W/O). The term “emulsions” means herein both the dispersions obtained in the absence of emulsifying surfactants and the emulsions obtained in the presence of emulsifying surfactants.

The nature of the compounds used for gelling the aqueous phase and their content in the composition are chosen as a function of the desired type of texture. The main hydrophilic thickeners or gelling agents which may be used in cosmetics are the following compounds:

crosslinked polymeric gelling agents such as the Carbopols (Goodrich) or crosslinked and at least partially neutralized 2-acrylamido-2-methylpropane sulphonic acid polymers such as, for example, the product sold under the name Hostacerin AMPS by the company Clariant. These gelling agents do not by themselves make it possible to stabilize an oily phase at a content of higher than 10% by weight, “by themselves” meaning in the absence of surfactants usually used in emulsions or in the absence of any other stabilizer, which limits the type of surfactant-free formulations that can be prepared, these conventional surfactant-free compositions being, however, desired for their satisfactory harmlessness with respect to the skin;

natural polymers such as xanthan gum and guar gum or cellulose derivatives, starches and alginates. These compounds are generally used in combination with crosslinked polymeric gelling agents since their thickening power is not large enough for them to be able to be used as sole gelling agents.

The stability of cosmetic compositions as a function of time and over a wide temperature range (5° C.-50° C.) is a crucial parameter to be taken into consideration during the development of a new composition. The presence of a liquid (emulsion) or solid (suspension) dispersed phase can induce destabilization phenomena such as sedimentation (deposition of solid), creaming (partial separation of the aqueous and oily phases) or aggregation and coalescence, this destabilization possibly being reflected at the microscopic and/or macroscopic level. One solution for limiting these destabilization phenomena consists in increasing the viscosity of the aqueous phase (continuous phase of the O/W emulsions) using the thickeners/gelling agents mentioned previously, which involves a high concentration of polymer in the composition, but this composition then has a film-forming and sticky feel after application to the skin. Moreover, for lower viscosities, the introduction of these thickeners/gelling agents may be insufficient to avoid the destabilization of the compositions.

In addition, cosmetic compositions may also comprise additives of all kinds and especially solid particles such as pigments, mineral UV-screening agents or fillers. These solid particles can also destabilize emulsions and result in sedimentation phenomena over time.

Document WO-A-01/16187 (Rhodia) describes block copolymers comprising at least one block of water-soluble nature also comprising hydrophobic units, and at least one block of predominantly hydrophobic nature, which, when dissolved in water, form a viscoelastic gel. However, as indicated below, the polymers used according to the present invention are not described in the document and they produce results that cannot be obtained with the polymers described in the document.

Documents EP-A-1 279 398 and EP-A-1 287 395 describe cosmetic compositions containing diblock and triblock polymers, for obtaining stable compositions of a wide range of textures. However, it has been observed that the polymers specifically described in these documents do not make it possible to obtain satisfactorily stable compositions when they contain solid particles such as mentioned above.

### SUMMARY OF THE INVENTION

There is thus still a need for a compound for gelling the aqueous phase of an emulsion in order to obtain a broad range of textures, whilst also being easy to disperse in water, whose thickening/gelling power is reproducible, and which preferably, moreover, has both gelling and emulsifying properties, this compound furthermore making it possible to obtain a composition having good cosmetic properties and good stability, irrespective of the oils content and/or even in the presence of solid particles.

The inventor has discovered, unexpectedly, that certain block polymers as described herein achieve the aim of the invention. These block polymers are water-soluble or water-dispersible polymers of triblock structure B-A-B, or of diblock structure A-B, in which A is an ionic water-soluble polymer block and B is a hydrophobic polymer block, in which the amount of polymer block A is greater than or equal to 50% of the total weight of the polymer B-A-B, and greater than or equal to 60% of the total weight of the polymer A-B, and in which the hydrophobic block B has a glass transition temperature of greater than or equal to 70° C. These polymers have aqueous-phase-structuring properties, at weight concentrations of less than 20%, and they make it possible to obtain not only cosmetic and/or dermatological compositions characterized by excellent stability, even for emulsions comprising more than 10% oily phase, which makes it possible to have a wide range of textures with good, e.g., cosmetic properties, but also compositions containing solid particles that are stable over time. These polymers are easy to dissolve or disperse in water and the gelling properties obtained are reproducible from one batch to another.

### DETAILED DESCRIPTION OF THE PREFERRED EMBODIMENTS

One subject of the present invention is a composition for topical application, wherein it comprises at least one aqueous phase comprising water and at least one water-soluble or water-dispersible polymer chosen from (1) polymers of triblock structure B-A-B in which A is an ionic water-soluble polymer block and B is a hydrophobic polymer block, in which the amount of polymer block A is greater than or equal to 50% of the total weight of the triblock polymer B-A-B, and in which the hydrophobic block B has a glass transition temperature of greater than or equal to 70° C., and/or (2) polymers of diblock structure A-B, in which A is an ionic water-soluble polymer block and B is a hydrophobic polymer block, in which the amount of polymer block A is greater than or equal to 60% of the total weight of the diblock polymer A-B and in which the hydrophobic block B has a glass transition temperature of greater than or equal to 70° C.

The expression “topical application” means herein an external application to keratin materials, i.e., especially, the skin, the scalp, the eyelashes, the eyebrows, the nails, mucous membranes and the hair.

Since the composition according to the invention is intended for topical application, it preferably contains a physiologically acceptable medium, i.e. a medium that is compatible with all keratin materials such as the skin, the nails, mucous membranes and the hair or any other area of body skin. The compositions according to the invention may especially constitute cosmetic or dermatological compositions.

In the present patent application, the expression “water-soluble or water-dispersible” means polymers which, when introduced into an aqueous phase at 25° C., at a weight concentration equal to 1%, allow the production of a macroscopically homogeneous and transparent solution, i.e. a solution with a maximum light transmittance value, at a wavelength equal to 500 nm, through a sample 1 cm thick, of at least 70% and preferably of at least 80%.

In the present patent application, the expression “polymer block” means a block polymer whose molar weight is greater than 400 g/mol and preferably greater than 800 g/mol.

In the present patent application, the expression “hydrophobic block” means a block polymer which, when introduced into a hydrocarbon solvent at 25° C., at a weight concentration equal to 1%, allows the production of a macroscopically homogeneous and transparent solution, i.e. a solution with a maximum light transmittance value, at a wavelength equal to 500 nm, through a sample 1 cm thick, of at least 70% and preferably of at least 80%. The hydrocarbon solvent used herein has a dielectric constant, measured at 25° C., of less than 50; this solvent may be chosen especially from alkanes such as cyclohexane (dielectric constant: 2.02); aromatic solvents such as ethylbenzene (dielectric constant: 2.4); ketones such as cyclohexanone (dielectric constant: 18.3); ethers such as diethyl ether (dielectric constant=4.4); alcohols such as cyclohexanol (dielectric constant: 15.0); chlorohydrocarbon solvents such as dichloromethane (dielectric constant: 9.08); amides such as dimethyl-formamide; and esters such as ethylacetate (dielectric constant: 6.02).

The expression “glass transition temperature” means the temperature above which an amorphous polymer becomes softer, this temperature being below the melting point (Polymer Handbook, 3rd edition, J. Brandrup, E. H. Immergut, J. Wiley & Sons, 1989). The glass transition phenomenon is reflected by changes in the properties of the polymer, such as the specific heat. The glass transition temperature may thus be evaluated by differential thermal analysis, according to ISO standard 11357-2.

The glass transition temperature is dependent on the molecular weight. Thus, the molecular weight of a polystyrene whose glass transition temperature is equal to 70° C. is 3 340 g/mol [value calculated from the following relationship given in the “Polymer Handbook”, Third Edition, published by J. Brandrup, E. H. Immergut, 1989, page V/82: Tg (K)=373−(100 000/M)].

The polymers used according to the invention allow satisfactory stabilization of the aqueous phases of compositions for topical application, especially cosmetic or dermatological compositions, containing solid particles.

A subject of the invention is also the use of at least one water-soluble or water-dispersible polymer chosen from (1) polymers of triblock structure B-A-B in which A is an ionic water-soluble polymer block and B is a hydrophobic polymer block, in which the amount of polymer block A is greater than or equal to 50% of the total weight of the triblock polymer B-A-B, and in which the hydrophobic block B has a glass transition temperature of greater than or equal to 70° C., and/or (2) polymers of diblock structure A-B, in which A is an ionic water-soluble polymer block and B is a hydrophobic polymer block, in which the amount of polymer block A is greater than or equal to 60% of the total weight of the diblock polymer A-B, and in which the hydrophobic block B has a glass transition temperature of greater than or equal to 70° C., to stabilize a cosmetic and/or dermatological composition comprising at least one aqueous phase and solid particles.

Moreover, the polymers used in the composition according to the invention have emulsifying properties and allow the preparation of an emulsion free of emulsifying surfactant, i.e. containing no or containing only small amounts of emulsifying surfactant (from 0 to about 1% by weight relative to the total weight of the composition, and preferably less than 0.5% by weight), especially of O/W emulsions. In the present patent application, the term “emulsifying surfactant” means monomeric surfactants with emulsifying properties.

Triblock polymer; diblock polymer

The polymers used according to the present invention differ from those described in document WO-A-01/16187 by the fact that they comprise a well-defined proportion of polymer block A (for the triblock polymer: greater than or equal to 50% of the total weight of the triblock polymer, and for the diblock polymer: greater than or equal to 60% in the diblock polymer, which corresponds to proportions of hydrophobic block of less than 50% and 40%, respectively), whereas, in WO-A-01/16187 the weight proportion between the predominantly hydrophilic and hydrophobic blocks is between 95/5 and 20/80.

In Examples 1 to 6 of WO-A-01/16187 concerning diblock polymers, the proportion of hydrophobic block is greater than 40%. Moreover, the diblock and triblock polymers having a weight proportion of hydrophobic block of less than 50% for the triblock polymers or less than 40% for the diblock polymers, given as examples in the document (Examples 7, 10 and 11), have a glass transition temperature of much less than 70° C., as shown in the table below.

| | Hydrophobic block (block B) | | | |
|---|---|---|---|---|
| | | Molar | | |
| | | weight | | |
| | | (g/mol) | Weight proportion | |
| | | of the | in the copolymer | |
| Example | Nature | block | (%) | Tg (° C.) |
| 1 | styrene | 5 900 | 45.7 | not |
| | methacrylic acid | | (block A = 54.3%) | calculated |
| | 2-hydroxyethyl | | | |
| | methacrylate | | | |
| 2 | styrene | 6 500 | 48.1 | not |
| | methacrylic acid | | (block A = 50.9%) | calculated |
| 3 | styrene | 6 400 | 50.6 | not |
| | 2-hydroxyethyl | | (block A = 49.4%) | calculated |
| | methacrylate | | | |
| 4 | styrene | 6 300 | 47.4 | not |
| | methacrylic acid | | (block A = 52.6%) | calculated |
| 5 | styrene | 6 900 | 51.3 | not |
| | methacrylic acid | | (block A = 48.7%) | calculated |
| | 2-hydroxyethyl | | | |
| | methacrylate | | | |
| 6 | styrene | 7 400 | 53.5 | not |
| | acrylic acid | | (block A = 46.5%) | calculated |
| 7 | styrene | 2 600 | 15.4 | 62 |
| | | | (block A = 84.6%) | |
| 10 | 98% (by weight) | 2 000 | 9.8 | 50 |
| | styrene | | (block A = 90.2%) | |
| | 2% methacrylic | | | |
| | acid | | | |
| 11 | 98% (by weight) | 2 000 | 9.8 | 50 |
| | styrene | | | |
| | 2% by weight | 500 | 2.5 | <50 |
| | methacrylic acid | | | |

Thus, the examples described in WO-A-01/16187 are different from the polymers used according to the invention, and not suggestive thereof, since:

either the weight proportion of the hydrophilic block is less than 60% (Examples 1 to 6),

or the hydrophobic block has a glass transition temperature of less than 70° C. (Examples 7, 10 and 11).

Moreover, the polymers in document WO-A-01/16187 are described for their water-gelling properties but are not described as having emulsifying power.

The weight (weight) proportion of the ionic hydrophilic block A in the diblock polymers A-B of the invention is greater than or equal to 60%, preferably greater than 60% and better still greater than 70%, relative to the total weight of the blocks A and B.

The weight (weight) proportion of the ionic hydrophilic block A in the triblock polymers B-A-B of the invention is greater than or equal to 50%, preferably greater than 50% and better still preferably greater than 60%, relative to the total weight of the blocks A and B.

The ionic water-soluble block A of the polymers of the invention is obtained from one or more water-soluble monomers (Ia) or the salts thereof, such as, for example:

(meth)acrylic acid,

styrenesulphonic acid,

vinylsulphonic acid and (meth)allylsulphonic acid,

vinylphosphonic acid,

maleic acid,

itaconic acid,

crotonic acid,

methyldiallylammonium chloride,

dimethylvinylimidazolium chloride,

ethylenic carboxybetaines or sulphobetaines obtained, for example, by quaternization of ethylenically unsaturated monomers including an amine function, with sodium salts of carboxylic acids containing a labile halogen (e.g.: chloroacetate) or with cyclic sulphones (e.g.: propanesulphone),

water-soluble vinyl monomers of formula (I) below:

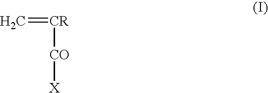

in which:

R is chosen from H, —CH₃, —C₂H₅ and —C₃H₇;

X is chosen from:

alkyl oxides of the type —OR₁ in which R₁ is a linear or branched, saturated or unsaturated hydrocarbon radical containing from 1 to 6 carbon atoms, substituted with at least one sulphonic (—SO₃⁻) and/or sulphate (—SO₄⁻) and/or phosphate (—PO₄H₂⁻) group and/or an ammonium (—N⁺R₂R₃R₄), the radicals R₂, R₃ and R₄ being, independently of each other, a linear or branched, saturated or unsaturated hydrocarbon radical containing 1 to 6 carbon atoms, with the proviso that the sum of the carbon atoms of R₁+R₂+R₃+R₄ does not exceed 6. The radical R₁ is optionally substituted with a halogen atom (iodine, bromine, chlorine or fluorine); hydroxyl (—OH); ether (—O—); primary amine (—NH₂); secondary amine (—NHR′); tertiary amine (—NR′R″), the radicals R′ and R″ being, independently of each other, a linear or branched, saturated or unsaturated hydrocarbon radical containing 1 to 6 carbon atoms, with the proviso that the sum of the carbon atoms of R₁+R′+R₆ does not exceed 6. An example of vinyl monomers including ester groups (X=OR₁) that may be mentioned is quaternized dimethylaminoethyl methacrylate (DMAEMA).

groups —NH₂, NHR₅ and —NR₅R₆ in which R₅ and R₆ are, independently of each other, a linear or branched, saturated or unsaturated hydrocarbon radical containing 1 to 6 carbon atoms, with the proviso that the total number of carbon atoms of R₅+R₆ does not exceed 6, the radicals R₅ and/or R₆ being substituted with at least one sulphonic (—SO₃⁻) and/or sulphate (—SO₄⁻) and/or phosphate (—PO₄H₂⁻) and/or quaternary amine (—N⁺R₇R₈R₉) group, the radicals R₇, R₈ and R₉ being, independently of each other, a linear or branched, saturated or unsaturated hydrocarbon radical containing 1 to 6 carbon atoms, with the proviso that the sum of the carbon atoms of R₅+R₆+R₇+R₈+R₉ does not exceed 6. The radicals R₅ and/or R₆ may optionally be substituted with a halogen atom (iodine, bromine, chlorine or fluorine); a hydroxyl (—OH); ether (—O—); primary amine (—NH₂); secondary amine (—NHR′); or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₅+R₆+R′+R″ does not exceed 6. Examples of vinyl monomers including amide groups that may be mentioned are 2-acrylamido-2-methyl-propanesulphonic acid (AMPS) and (meth)acrylamidopropyltrimethylammonium chloride (APTAC and MAPTAC).

In formula (I), the substituents R and X are such that the monomer of formula (I) is water-soluble.

Along with the water-soluble monomers (Ia) mentioned above and always present in the ionic water-soluble block A, the latter may also optionally contain one or more neutral water-soluble monomers (Ib) such as, for example:

(meth)acrylamide,

N-vinylacetamide and N-methyl-N-vinylacetamide,

N-vinylformamide and N-methyl-N-vinylformamide,

maleic anhydride,

vinylamine,

N-vinyllactams including a cyclic alkyl group containing from 4 to 9 carbon atoms, such as N-vinylpyrrolidone, N-butyrolactam and N-vinylcaprolactam,

vinyl alcohol of formula CH₂═CHOH,

water-soluble vinyl monomers of formula (II) below:

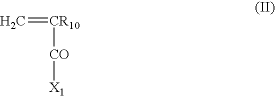

in which:

R₁₀ is chosen from H, —CH₃, —C₂H₅ and —C₃H₇;

X1 is chosen from:

alkyl oxides of the type —OR₁₁ in which R₁₁ is a linear or branched, saturated or unsaturated hydrocarbon radical containing from 1 to 6 carbon atoms, optionally substituted with a halogen atom (iodine, bromine, chlorine or fluorine); a hydroxyl (—OH); ether (—O—); primary amine (—NH₂); secondary amine (—NHR′) or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₁₁+R′+R″ does not exceed 6. Examples of monomers of formula (II) in which X₂ is a radical —OR₁₁ that may be mentioned include glycidyl (meth)acrylate, hydroxyethyl methacrylate and ethylene glycol (meth)acrylate, diethylene glycol (meth)acrylate and polyalkylene glycol (meth)acrylates.

groups —NH₂, —NHR₁₂ and —NR₁₂R₁₃ in which R₁₂ and R₁₃ are, independently of each other, linear or branched, saturated or unsaturated hydrocarbon radicals containing 1 to 6 carbon atoms, with the proviso that the total number of carbon atoms of R₁₂+R₁₃ does not exceed 6, the radicals R₁₂ and R₁₃ optionally being substituted with a halogen atom (iodine, bromine, chlorine or fluorine); a hydroxyl (—OH); ether (—O—); primary amine (—NH₂); secondary amine (—NHR′); or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₁₂+R₁₃+R′+R″ does not exceed 6. Examples of monomers of this type that may be mentioned include dimethylaminoethyl-methacrylamide.

In formula (II), the substituents R₁₀ and X₁are such that the monomer of formula (II) is water-soluble.

Along with the water-soluble monomers (Ia) mentioned above and always present, the ionic water-soluble block A may furthermore optionally be obtained from one or more hydrophobic monomers (Ic), the hydrophobic monomers being present in an amount that is low enough for the block A to be water-soluble. As examples of hydrophobic monomers (Ic) that may be used in the ionic water-soluble block A, mention may be made of:

styrene and its derivatives such as 4-butyl-styrene, α-methylstyrene and vinyltoluene;

vinyl acetate of formula CH₂═CH—OCOCH₃;

vinyl ethers of formula CH₂═CHOR₁₄ in which R₁₄ is a linear or branched, saturated or unsaturated hydrocarbon radical containing from 1 to 6 carbon atoms;

acrylonitrile;

caprolactone;

vinyl chloride and vinylidene chloride;

silicone derivatives, leading, after polymerization, to silicone polymers such as methacryloxypropyltris(trimethylsiloxy)silane and silicone methacrylamides,

hydrophobic vinyl monomers of formula (III) below:

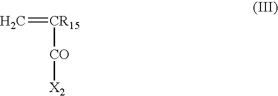

in which:

R₁₅ is chosen from H, —CH₃, —C₂H₅ and —C₃H₇;

X₂ is chosen from:

alkyl oxides of the type —OR₁₆ in which R₁₆ is a linear or branched, saturated or unsaturated hydrocarbon radical containing from 1 to 6 carbon atoms.

groups —NH₂, —NHR₁₇ and —NR₁₇R₁₈ in which R₁₇ and R₁₈ are, independently of each other, linear or branched, saturated or unsaturated hydrocarbon radicals containing 1 to 6 carbon atoms, with the proviso that the total number of carbon atoms of R₁₇+R₁₈ does not exceed 6. Examples of monomers of formula (III) that may be mentioned are methyl methacrylate, ethyl methacrylate, n-butyl (meth)acrylate, tert-butyl (meth)acrylate, cyclohexyl acrylate and isobornyl acrylate.

In addition to the water-soluble monomers mentioned above, the ionic water-soluble block A may also be an ionic water-soluble polymer such as, for example, polyethyleneimine.

The ionic water-soluble polymer block A may be made from any mixture of the above materials and is preferably totally or partially neutralized with a mineral or organic base. The expression “partially neutralized” means a neutralization of at least 20 mol %. The ionic water-soluble block A may be neutralized with a mineral or organic base. The neutralization base may be chosen, for example, from sodium hydroxide, sodium, ammonium, lithium, calcium or magnesium salts, ammonium salts substituted with 1 to 4 alkyl groups bearing from 1 to 15 carbon atoms, or also monoethanolamine, diethanolamine, triethanolamine, aminoethylpropanediol or N-methylglucamine, and basic amino acids, such as arginine and lysine, and mixtures thereof.

The chemical nature and the molar weight of the hydrophobic block B of the polymers of the invention are chosen such that they have a glass transition temperature greater than or equal to 70° C.

The hydrophobic block B may be obtained from one or more hydrophobic monomers (Id) such as, for example:

styrene and its derivatives such as 4-butyl-styrene, α-methylstyrene and vinyltoluene,

vinyl acetate of formula CH₂═CH—OCOCH₃,

vinyl ethers of formula CH₂═CHOR₁₉ in which R₁₉ is a linear or branched, saturated or unsaturated hydrocarbon radical containing from 1 to 6 carbons,

acrylonitrile,

vinyl chloride and vinylidene chloride,

caprolactone,

alkenes such as ethylene, propylene, butylene and butadiene,

silicone derivatives, leading, after polymerization, to silicone polymers such as polydimethylsiloxane, methacryloxypropyltris(trimethyl-siloxy)silane and silicone methacrylamides,

hydrophobic vinyl monomers of formula (IV) below:

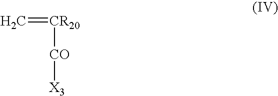

in which:

R₂₀ is chosen from H, —CH₃, —C₂H₅ or —C₃H₇;

X₃ is chosen from:

alkyl oxides of the type —OR₂₁ in which R₂₁ is a linear or branched, saturated or unsaturated hydrocarbon radical containing from 1 to 22 carbon atoms, optionally substituted with a halogen atom (iodine, bromine, chlorine or fluorine); a sulphonic (—SO₃⁻), sulphate (—SO₄⁻), phosphate (—PO₄H₂⁻), hydroxyl (—OH), ether (—O—), primary amine (—NH₂), secondary amine (—NHR₂₂), tertiary amine (—NR₂₂R₂₃) or quaternary ammonium (—N⁺R₂₂R₂₃R₂₄) group, the radicals R₂₂, R₂₃ and R₂₄ being, independently of each other, a linear or branched, saturated or unsaturated hydrocarbon radical containing 1 to 22 carbon atoms, with the proviso that the sum of the carbon atoms of R₂₁+R₂₂+R₂₃+R₂₄ does not exceed 22. As examples of hydrophobic vinyl monomers including alkyl oxide groups of the type —OR₂₁, mention may be made of methyl methacrylate, ethyl methacrylate, n-butyl (meth)acrylate, tert-butyl (meth)acrylate, cyclohexyl acrylate, isobornyl acrylate and 2-ethylhexyl acrylate. R₂₁ can also be a perfluoroalkyl radical preferably containing from 1 to 18 carbon atoms: examples of monomers of formula (IV) with a perfluoroalkyl radical constituting the group R₂₁ that may be mentioned include ethylperfluorooctyl acrylate and trifluoromethyl (meth)acrylate.

groups —NH₂, —NHR₂₅ and —NR₂₅R₂₆ in which the radicals R₂₅ and R₂₆ are, independently of each other, a linear or branched, saturated or unsaturated hydrocarbon radical containing 1 to 22 carbon atoms, with the proviso that the total number of carbon atoms of R₂₅+R₂₆ does not exceed 22, the radicals R₂₅ and R₂₆ optionally being substituted with a halogen atom (iodine, bromine, chlorine or fluorine); a hydroxyl (—OH), ether (—O—), carboxylate (—CO₂⁻), sulphonic (—SO₃⁻), sulphate (—SO₄⁻), phosphate (—PO₄H₂⁻), carboxylic acid (—COOH), primary amine (—NH₂), secondary amine (—NHR′), tertiary amine (—NR′R″) or quaternary amine (—N⁺R′R″R′″) group, the radicals R′, R″ and R′″ representing, independently of each other, a linear or branched, saturated or unsaturated hydrocarbon radical containing 1 to 22 carbon atoms, with the proviso that the sum of the carbon atoms of R₂₅+R₂₆+R′+R″+R′″ does not exceed 22. R₂₅ and R₂₆, independently of each other, may also be a perfluoroalkyl radical preferably containing from 1 to 18 carbon atoms.

In formula (IV), the substituents R₂₀ and X₃ are such that the monomer of formula (IV) is hydrophobic.

Along with the hydrophobic monomers (Id) mentioned above and always present in the hydrophobic block B, the latter may furthermore optionally be obtained from one or more water-soluble monomers, the water-soluble monomers being present in an amount that is low enough for the block B to be hydrophobic. Examples of water-soluble monomers (Ie) that may be mentioned include the following compounds, or salts thereof:

(meth)acrylic acid;

styrenesulphonic acid;

vinylsulphonic acid and (meth)allylsulphonic acid;

vinylphosphonic acid;

maleic acid and anhydride;

itaconic acid;

crotonic acid;

dimethyldiallylammonium chloride;

methylvinylimidazolium chloride;

(meth)acrylamide;

N-vinylacetamide and N-methyl-N-vinylacetamide;

N-vinylformamide and N-methyl-N-vinylformamide;

N-vinyllactams including a cyclic alkyl group containing from 4 to 9 carbon atoms, such as N-vinyl-pyrrolidone, N-butyrolactam and N-vinylcaprolactam,

vinyl alcohol of formula CH₂═CHOH;

2-vinylpyridine and 4-vinylpyridine;

the water-soluble vinyl monomers of formula (V) below:

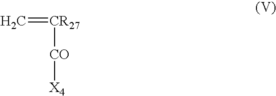

in which:

R₂₇ is chosen from H, —CH₃, —C₂H₅ and —C₃H₇;

X₄ is chosen from:

alkyl oxides of the type —OR₂₈ in which R₂₈ is a linear or branched, saturated or unsaturated hydrocarbon radical containing from 1 to 6 carbon atoms, optionally substituted with a halogen atom (iodine, bromine, chlorine or fluorine); a sulphonic (—SO₃⁻), sulphate (—SO₄⁻), phosphate (—PO₄H₂⁻), hydroxyl (—OH), ether (—O—), primary amine (—NH₂), secondary amine (—NHR′), tertiary amine (—NR′R″) or a quaternary amine (—N⁺R′R″R′″) group, the radicals R′, R″ and R′″ being, independently of each other, a linear or branched, saturated or unsaturated hydrocarbon radical containing 1 to 6 carbon atoms, with the proviso that the sum of the carbon atoms of R₂₈+R′+R″+R′″ does not exceed 6. Examples of vinyl monomers including ester groups (X₄═OR₂₈) that may be mentioned include quaternized dimethylaminoethyl methacrylate (DMAEMA), glycidyl (meth)acrylate, hydroxyethyl methacrylate and ethylene glycol (meth)acrylate, diethylene glycol (meth)acrylate or polyalkylene glycol (meth)acrylates.

groups —NH₂, —NHR₂₉ and —NR₂₉R₃₀ in which the radicals R₂₉ and R₃₀ are, independently of each other, linear or branched, saturated or unsaturated hydrocarbon radicals containing 1 to 6 carbon atoms, with the proviso that the total number of carbon atoms of R₂₉+R₃₀ does not exceed 6, the radicals R₂₉ and/or R₃₀ optionally being substituted with a halogen atom (iodine, bromine, chlorine or fluorine); a sulphonic (—SO₃⁻); sulphate (SO₄⁻); phosphate (—PO₄H₂⁻); hydroxyl (—OH); ether (—O—); primary amine (—NH₂); secondary amine (—NHR′); tertiary amine (—NR′R″) or quaternary amine (—N⁺R′R″R′″) group, the radicals R′, R″ and R′″ being, independently of each other, a linear or branched, saturated or unsaturated hydrocarbon radical containing 1 to 6 carbon atoms, with the proviso that the sum of the carbon atoms of R₂₉+R₃₀+R′+R″+R′″ does not exceed 6. Examples of water-soluble vinyl monomers of formula (V) including amide groups that may be mentioned are 2-acrylamido-2-methylpropanesulphonic acid (AMPS), (meth)acrylamidopropyltrimethylammonium chloride (APTAC and MAPTAC) and N,N-dimethylacrylamide.

In formula (V), the substituents R₂₇ and X₄ are such that the monomer of formula (V) is water-soluble.

The hydrophobic block B of the polymers of the invention may be obtained from several monomers, including any or all of those described above, the proportion of rigid monomers being large enough for the glass transition temperature of the block B to be greater than or equal to 70° C.

The polymers A-B and B-A-B used according to the invention have a molar weight preferably ranging from 1 000 g/mol to 500 000 g/mol and more preferably from 2 000 g/mol to 100 000 g/mol. For the diblock polymers, the ionic water-soluble block A preferably has a molar weight ranging from 600 g/mol to 300 000 g/mol and more preferably from 1 200 g/mol to 60 000 g/mol and the hydrophobic block B has a molar weight preferably ranging from 400 g/mol to 200 000 g/mol and more preferably from 800 g/mol to 40 000 g/mol. For the triblock polymers, the ionic water-soluble block A has a molar weight preferably ranging from 500 g/mol to 250 000 g/mol and more preferably from 1 000 g/mol to 50 000 g/mol, and the hydrophobic block B has a molar weight preferably ranging from 500 g/mol to 250 000 g/mol and more preferably from 1 000 g/mol to 50 000 g/mol.

The (diblock and triblock) block polymers used in the composition of the invention may be prepared by synthetic processes conventionally used to obtain block polymers. Such preparation is within the skill of the ordinary artisan in view of this disclosure. Examples of preparation processes that may be mentioned include polymerizations of anionic or cationic type and controlled free-radical polymerization, which may be carried out according to various processes such as, for example, the atom transfer approach (Atom Transfer Radical Polymerization or ATRP) (see JACS, 117, page 5614 (1995)), the method via radicals such as nitroxides (see Georges et al., Macromolecules, 1993, 26, 2987) or the reversible chain transfer approach with addition-fragmentation (Radical Addition-Fragmentation chain Transfer) such as the MADIX process (Macromolecular Design via the Interchange of Xanthate) (see Charmot D., Corpart P., Adam H., Zard S. Z., Biadatti T., Bouhadir G., Macromol. Symp., 2000, 150, 23). These synthetic processes may be used to obtain the blocks A and B of the polymers of the invention; they may also be used to synthesize only one of the two blocks of the polymer of the invention, the other block being introduced into the final polymer by means of the primer used or alternatively by a coupling reaction between the blocks A and B.

The amount of block polymer(s) in the composition of the invention can vary depending on the type of composition that it is desired and the desired degree of gelation. It can range, for example, from 0.01% to 20% by weight, preferably from 0.05% to 20% by weight and better still from 0.1% to 15% by weight relative to the total weight of the composition.

The cosmetic or dermatological compositions of the invention may have a pH that varies within a wide range and that may be, for example, from 2 to 10, preferably from 3 to 8 and better still from 4 to 7.

The aqueous phase of the compositions according to the invention comprises at least water. The compositions of the invention may contain, in addition to water, at least one oily phase and/or one or more hydrophilic, lipophilic and/or amphiphilic organic solvents that are physiologically acceptable, i.e. well tolerated and that give a cosmetically acceptable feel. The amount of aqueous phase can range, for example, from 10% to 99.99% by weight, preferably from 20% to 95% by weight and better still from 30% to 90% by weight relative to the total weight of the composition. Water can represent all or part of the aqueous phase.

The organic solvents can represent, e.g., from 5% to 50% of the total weight of the composition. These solvents may be chosen for example from the group consisting of hydrophilic organic solvents, lipophilic organic solvents and amphiphilic solvents, or mixtures thereof.

Included among the organic solvents, for example, are linear or branched lower monoalcohols containing from 1 to 8 carbon atoms, for instance ethanol, propanol, butanol, isopropanol or isobutanol; polyols such as propylene glycol, isoprene glycol, butylene glycol, glycerol or sorbitol; monoalkyl or dialkyl isosorbide in which the alkyl groups contain from 1 to 5 carbon atoms, for instance dimethyl isosorbide; polyethylene glycols, especially those containing from 6 to 80 ethylene oxides; ethylene glycol ethers, for instance diethylene glycol monomethyl or monoethyl ether; propylene glycol ethers, for instance dipropylene glycol methyl ether; polyol esters and ethers, such as polypropylene glycol (PPG) esters and more especially fatty acid esters of polypropylene glycol (PPG), fatty alkyl ethers of PPG, for instance PPG-23 oleyl ether and PPG-36 oleate; fatty acid alkyl esters, such as diisopropyl adipate, dioctyl adipate or alkyl benzoates; and mixtures thereof.

The composition of the invention may comprise at least one fatty phase, also known as an oily phase. The oily phase may represent, for example, from 0% to 80% by weight relative to the total weight of the composition. In an emulsion, the oily phase may represent, for example, from 0.1% to 50% by weight, preferably from 1% to 40% by weight and better still from 5% to 30% by weight relative to the total weight of the composition.

The fatty phase or oily-phase usually contains at least one oil, especially a cosmetic oil. As oils that may be used in the composition of the invention, mention may be made, for example, of:

hydrocarbon-based oils of animal origin, such as perhydrosqualene;

hydrocarbon-based oils of plant origin, such as liquid triglycerides of fatty acids containing from 4 to 10 carbon atoms, for instance heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides, for instance those sold by the company Stearineries Dubois or those sold under the names Miglyol 810, 812 and 818 by the company Dynamit Nobel, jojoba oil or karite butter oil;

synthetic esters and synthetic ethers, especially of fatty acids, for instance oils of formulae R^{a}COOR^{b} and R^{a}OR^{b} in which R^{a} represents a fatty acid residue containing from 8 to 29 carbon atoms and R^{b} represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as Parleam® oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

alkoxylated and especially ethoxylated fatty alcohols such as oleth-12, ceteareth-12 and ceteareth-20;

partially hydrocarbon-based and/or silicone-based fluoro oils, for instance those described in document JP-A-2 295 912. Examples of fluoro oils which may also be mentioned include perfluoromethylcyclopentane and perfluoro-1,3-dimethylcyclohexane, sold under the names “Flutec PC1®” and “Flutec PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoroethoxyisobutane; perfluoromorpholine derivatives, such as the 4-trifluoromethylperfluoromorpholine sold under the name “PF 5052®” by the company 3M;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendant or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyldimethicones, diphenylmethyldiphenyltrisiloxanes, 2-phenylethyltrimethylsiloxysilicates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin waxes, lignite wax or microcrystalline waxes, ceresin or ozokerite, synthetic waxes such as polyethylene waxes, Fischer-Tropsch waxes; gums such as silicone gums (dimethiconol); silicone resins such as trifluoromethyl-C1-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries.

These fatty substances may be chosen in a varied manner by a person skilled in the art so as to prepare a composition having the desired properties, for example in terms of consistency or texture.

The composition of the invention may comprise an aqueous phase alone, or an aqueous phase and a fatty phase (W/O or O/W emulsion) or several aqueous phases and one fatty phase (W/O/W emulsion) or one aqueous phase and several fatty phases (O/W/O emulsion). It may thus constitute a solution, a gel or an emulsion (simple or multiple).

When the composition is an emulsion, it may contain an emulsifying surfactant, but, according to one particular embodiment of the invention, it may contain virtually no emulsifying surfactant, i.e. less than 0.5% by weight of emulsifying surfactant relative to the total weight of the composition, and may even be free of any emulsifying surfactant.

As examples of emulsifying surfactants for W/O emulsions, mention may be made, for example, of dimethicone copolyols such as the mixture of cyclomethicone and of dimethicone copolyol, sold under the name “DC 5225 C” by the company Dow Corning, and alkyldimethicone copolyols such as laurylmethicone copolyol sold under the name “Dow Corning 5200 Formulation Aid” by the company Dow Corning and cetyldimethicone copolyol sold under the name Abil EM 90® by the company Goldschmidt, or the polyglyceryl-4 isostearate/cetyldimethicone copolyol/hexyl laurate mixture sold under the name Abil WE 09 by the company Goldschmidt. One or more co-emulsifiers may also be added thereto. Advantageously, the co-emulsifier may be chosen from the group comprising alkyl esters of polyol. As alkylesters of polyol, mention may be made especially of glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

For the O/W emulsions, examples of emulsifiers that may be mentioned include nonionic surfactants, especially such as polyol esters of fatty acids with a saturated or unsaturated chain containing, for example, from 8 to 24 carbon atoms and better still from 12 to 22 carbon atoms, and oxyalkylene derivatives thereof, i.e. derivatives comprising oxyethylene and/or oxypropylene units, for instance glyceryl esters of C₈-C₂₄ fatty acids, and oxyalkylene derivatives thereof; polyethylene glycol esters of C₈-C₂₄ fatty acids, and oxyalkylene derivatives thereof; sorbitol esters of C₈-C₂₄ fatty acids and oxyalkylene derivatives thereof; sugar (sucrose, glucose or alkylglucose) esters of C8-C₂₄ fatty acids, and oxyalkylene derivatives thereof, fatty alcohol ethers, sugar ethers of C₈-C₂₄ fatty alcohols, and mixtures thereof.

In a known manner, the compositions of the invention can contain adjuvants that are common in cosmetics and dermatology, standard hydrophilic or lipophilic thickeners and/or gelling agents; polymers; moisturizers; emollients; hydrophilic or lipophilic active agents; free-radical scavengers; sequestering agents; antioxidants; preserving agents; acidifying or basifying agents; fragrances; pigments; fillers; film-forming agents; dyestuffs and mixtures thereof. The amounts of these various adjuvants are those conventionally used in the fields under consideration. Needless to say, a person skilled in the art will take care to select the optional adjuvants added to the composition according to the invention such that the advantageous properties intrinsically associated with the composition in accordance with the invention are not, or are not substantially, adversely affected by the envisaged addition.

Examples of gelling agents that may be mentioned include hydrophilic polymers such as carboxyvinyl polymers, for instance carbomers; 2-acrylamido-2-methylpropanesulphonic acid polymers that are soluble or dispersible in aqueous phase, for instance the polymer sold under the name “Hostacerin AMPS” by the company Clariant; synthetic neutral polymers, for instance polyvinylpyrrolidone (PVP) or polyvinyl acetate (PVA); polysaccharides, for instance guar gum, xanthan gum and cellulose derivatives such as, for example, hydroxyethylcellulose; water-soluble or water-dispersible silicone derivatives, for instance acrylic silicones and cationic silicones. Lipophilic gelling agents, such as modified clays or modified polysaccharides, may also be used.

As active agents that may be used in the composition of the invention, examples that may be mentioned include moisturizers and, for example, protein hydrolysates and polyols such as glycerol, glycols, for instance polyethylene glycols, and sugar derivatives; natural extracts; anti-inflammatory agents; calming agents; depigmenting agents; procyannidol oligomers; vitamins, for instance vitamin A (retinol), vitamin C (ascorbic acid), vitamin E (tocopherol), vitamin B5 (panthenol), vitamin B3 (niacinamide), derivatives of these vitamins (especially esters) and mixtures thereof; urea; caffeine; depigmenting agents such as cojic acid, hydroquinone and caffeic acid; salicylic acid and its derivatives; α-hydroxy acids such as lactic acid and glycolic acid and derivatives thereof; retinoids such as carotenoids and vitamin A derivatives; sunscreens; hydrocortisone; melatonin; algal, fungal, plant, yeast or bacterial extracts; plant proteins; polysaccharides of plant origin in microgel or other form; wax dispersions; mixed silicates; enzymes; DHEA and its derivatives and metabolites; antibacterial active agents, for instance 2,4,4′-trichloro-2′-hydroxydiphenyl ether (or Triclosan), 3,4,4′-trichlorocarbanilide (or Triclocarban) and the acids indicated above and especially salicylic acid and its derivatives; matt-effect agents, for instance fibres; agents for preventing hair loss and/or for promoting hair regrowth; anti-wrinkle agents; tensioning agents such as synthetic polymers; and mixtures thereof.

The UV-screening agents or photoprotective agents that may be used in the composition in accordance with the invention include: organic photoprotective agent and/or at least one mineral photoprotective agent, active in the UVA and/or UVB range (absorbers), which are water-soluble or liposoluble or else insoluble in the cosmetic solvents commonly used.

The organic photoprotective agents are especially chosen from anthranilates; cinnamic derivatives; dibenzoylmethane derivatives; salicyclic derivatives; camphor derivatives; triazine derivatives such as those described in patent applications U.S. Pat. No. 4,367,390, EP-A-863 145, EP-A-517 104, EP-A-570 838, EP-A-796 851, EP-A-775 698, EP-A-878 469, EP-A-933 376, EP-A-507 691, EP-A-507 692, EP-A-790 243 and EP-A-944 624; benzophenone derivatives; β,β-diphenylacrylate derivatives; benzotriazole derivatives; benzalmalonate derivatives; benzimidazole derivatives; imidazolines; bis-benzazolyl derivatives as described in patents EP-A-669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB-A-2 303 549, DE-A-197 26 184 and EP-A-893 119; screening polymers and screening silicones such as those described especially in patent application WO-A-93/04665; dimers derived from α-alkylstyrene such as those described in patent application DE-A-198 55 649.

As examples of UV-A-active and/or UV-B-active photoprotective agents, mention may be made of those given below under their INCI name:

para-aminobenzoic acid derivatives, including the following: PABA, Ethyl PABA, Ethyl dihydroxypropyl PABA, Ethylhexyl dimethyl PABA sold in particular under the name “Escalol 507” by ISP, Glyceryl PABA, PEG-25 PABA sold under the name “Uvinul P25” by BASF,

salicylic derivatives, including the following: Homosalate sold in particular under the name “Neo Heliopan OS” by Haarmann and Reimer, Dipropylene glycol salicylate sold in particular under the name “Dipsal” by Scher, TEA salicylate sold in particular under the name “Neo Heliopan TS” by Haarmann and Reimer,

dibenzoylmethane derivatives, including the following: Butyl methoxy-dibenzoylmethane sold in particular under the trade name “Parsol 1789” by Hoffmann LaRoche, Isopropyldibenzoylmethane,

cinnamic derivatives, including the following: Ethylhexyl methoxycinnamate sold in particular under the trade name “Parsol MCX” by Hoffmann LaRoche, Isopropyl methoxycinnamate, Isoamyl methoxycinnamate sold in particular under the trade name “Neo Heliopan E 1000” by Haarmann and Reimer, Cinoxate, DEA methoxycinnamate, Diisopropyl methylcinnamate, Glyceryl ethylhexanoate dimethoxycinnamate,

β,β′-diphenylacrylate derivatives, including the following: Octocrylene sold in particular under the trade name “Uvinul N539” by BASF, Etocrylene sold in particular under the trade name “Uvinul N35” by BASF,

benzophenone derivatives, including the following: Benzophenone-1 sold in particular under the trade name “Uvinul 400” by BASF, Benzophenone-2 sold in particular under the trade name “Uvinul D50” by BASF, Benzophenone-3 or Oxybenzone sold in particular under the trade name “Uvinul M40” by BASF, Benzophenone-4 sold in particular under the trade name “Uvinul MS40” by BASF, Benzophenone-5, Benzophenone-6 sold in particular under the trade name “Helisorb 11” by Norquay, Benzophenone-8 sold in particular under the trade name “Spectra-Sorb UV-24” by American Cyanamid, Benzophenone-9 sold in particular under the trade name “Uvinul DS-49” by BASF, Benzophenone-12, and n-hexyl 2-(4-diethylamino-2-hydroxybenzoyl) benzoate,

benzylidenecamphor derivatives, including the following: 3-Benzylidenecamphor, 4-Methyl-benzylidenecamphor sold in particular under the name “Eusolex 6300” by Merck, Benzylidenecamphorsulphonic acid, Camphor benzalkonium methosulphate, Terephthalylidenedicamphorsulphonic acid, Polyacrylamidomethylbenzylidenecamphor,

phenylbenzimidazole derivatives, including the following: Phenylbenzimidazolesulphonic acid sold in particular under the trade name “Eusolex 232” by Merck, Disodium phenyl dibenzimidazole tetrasulphonate sold in particular under the trade name “Neo Heliopan AP” by Haarmann and Reimer,

triazine derivatives, including the following: Anisotriazine sold in particular under the trade name “Tinosorb S” by Ciba Specialty Chemicals, Ethylhexyltriazone sold in particular under the trade name “Uvinul T150” by BASF, Diethylhexylbutamidotriazone sold under the trade name “Uvasorb HEB” by Sigma 3V, and 2,4,6-tris (4′aminodiisobutyl benzalmalonate)-s-triazine,

benzotriazole derivatives, including the following: Drometrizole trisiloxane sold under the name “Silatrizole” by Rhodia Chimie, Methylenebis(benzotriazolyl)tetramethylbutylphenol, sold in particular in solid form under the trade name “Mixxim BB/100” by Fairmount Chemical or in micronized form in aqueous dispersion under the trade name “Tinosorb M” by Ciba Specialty Chemicals,

anthranilic derivatives, including the following: Menthyl anthranilate sold under the trade name “Neo Heliopan MA” by Haarmann and Reimer,

imidazoline derivatives, including the following: Ethylhexyldimethoxybenzylidenedioxoimidazoline propionate,

benzalmalonate derivatives, including the following: Polyorganosiloxane containing benzalmalonate functions, sold under the trade name “Parsol SLX” by Hoffmann LaRoche,

and mixtures thereof.

The organic photoprotective agents that are more particularly preferred are chosen from the following compounds: ethylhexyl salicylate, ethylhexyl methoxycinnamate, octocrylene, phenylbenzimidazole sulphonic acid, benzophenone-3, benzophenone-4, benzophenone-5, 4-methylbenzylidene camphor, terephthalylidene dicamphor sulphonic acid, disodium phenyl dibenzimidazole tetrasulphonate, 2,4,6-tris(4′-amino diisobutylbenzalmalonate)-s-triazine, anisotriazine, ethylhexyl triazone, diethylhexyl butamido triazone, methylene bis-benzotriazolyl tetramethylbutylphenol, drometrizole trisiloxane, and mixtures thereof.

The mineral photoprotective agents may be chosen from pigments or nanopigments (mean size of the primary particles: generally between 5 nm and 100 nm and preferably between 10 nm and 50 nm) of coated or uncoated metal oxides, for instance nanopigments of titanium oxide (amorphous or crystallized in rutile and/or anatase form), of iron oxide, of zinc oxide, of zirconium oxide or of cerium oxide, which are all UV-photoprotective agents that are well known per se. Standard coating agents are, moreover, alumina and/or aluminium stearate. Such coated or uncoated metal oxide nanopigments are described in particular in patent applications EP-A-518 772 and EP-A-518 773. Examples that may be mentioned include the hydrophilic nanotitanium sold under the name Mirasun TIW60 by the company Rhodia and the lipophilic nanotitanium sold under the name MT100T by the company Tayca or UV Titan M 160 sold by the company Kemira.

The UV-screening agents are generally present in the compositions according to the invention in proportions ranging from 0.1% to 20% by weight relative to the total weight of the composition, and preferably ranging from 0.2% to 15% by weight relative to the total weight of the composition.

The composition may, without being destabilized, contain solid particles, for instance, without this list being exhaustive, physical sunblocks such as those indicated above, pigments and fillers, and mixtures thereof.

Pigments that may be mentioned include mineral pigments and especially metal oxides such as titanium dioxide, zirconium dioxide or cerium dioxide, and also zinc oxide, iron oxide or chromium oxide, ferric blue, nacres such as mica coated with titanium oxide, with iron oxide, with natural pigment or with bismuth oxychloride, and also coloured titanium mica; and organic pigments such as carbon black and lakes, which are calcium, barium, aluminium or zirconium salts of acidic dyes such as halo acid dyes, azo dyes or anthraquinone dyes.

These pigments may be treated so as to make their surface hydrophobic; this treatment may be carried out according to methods known to those skilled in the art; the pigments may especially be coated with silicone compounds such as PDMSs and/or with polymers.

Examples of fillers that may be mentioned, besides pigments, include silica powder; talc; polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as optionally crosslinked corn starch, wheat starch or rice starch, such as starch powders crosslinked with octenylsuccinic anhydride, sold under the name Dry-Flo by the company National Starch; silicone resin microbeads such as those sold under the name Tospearl by the company Toshiba Silicone; colloidal particles of mineral fillers such as colloidal silica nanoparticles, for instance the product sold under the name Cosmo S40 by the company Catalysts and Chemicals and the product sold under the name Ludox AM by the company Grace; and mixtures thereof.

The compositions according to the invention may be in the form of gels, lotions, milks, more or less unctuous creams, or pastes. These compositions are prepared according to the usual methods. Since the block polymer used is water-soluble, it is generally introduced in an aqueous phase.

The compositions of the invention may be used as care, treatment, protective, cleansing, makeup-removing and/or makeup products for keratin materials (skin, hair, scalp, eyelashes, eyebrows, nails or mucous membranes) such as protective, treatment or care creams for the face, for the hands or for the body, protective or care body milks, and gels or mousses to care for the skin and/or mucous membranes (lips).

The compositions of the invention can contain sunscreens and can thus also be used as antisun products.

The compositions may be used for makeup, especially for making up the skin, the eyebrows, the eyelashes and the lips, such as face creams, foundations, mascaras or lipsticks. Such products generally contain pigments.

The compositions according to the invention may also be used as rinse-out products or as leave-in products for cleansing facial skin and/or body skin and/or for cleansing the hair, for example as haircare products, including haircare and hair conditioning products.

A subject of the invention is the cosmetic use of a cosmetic composition as defined above, as a rinse-out or leave-in hair product.

A subject of the invention is the cosmetic use of a cosmetic composition as defined above, as a product for cleansing and/or removing makeup from the skin and/or the eyes.

A subject of the invention is also the cosmetic use of a cosmetic composition as defined above, as a care product for the skin, the hair, the scalp, the eyelashes, the eyebrows, the nails or mucous membranes.

A subject of the invention is also the cosmetic use of a cosmetic composition as defined above, as a makeup product.

A subject of the invention is also the cosmetic use of a cosmetic composition as defined above, as an antisun product (for protection against sunlight and/or the UV radiation of tanning machines).

Another subject of the invention is a (non-therapeutic) cosmetic process for treating a keratin material such as the skin, the scalp, the hair, the eyelashes, the eyebrows, the nails or the mucous membranes, wherein a cosmetic composition as defined above is applied to the keratin material.

### EXAMPLES

The examples that follow serve to illustrate the invention without, however, being limiting in nature. The amounts indicated are in weight percentages, except where otherwise mentioned.

### Example 1

### Aqueous Dispersions of Nanotitanium Oxide (5%) Comprising 2% (by Weight) of a Hydrophilic Gelling Agent

The gelling power of the polymers was demonstrated by rheology using a rheometer of RS150 type (Haake) operating under controlled stress, and equipped with a 35 mm/2° cone/plate geometry. A Peltier-effect temperature control system makes it possible to maintain the sample at a temperature of 20° C. during the measurements. The Theological characterizations were performed in flow and dynamic modes.

Flow Measurements:

The measurements are carried out by imposing an ascending stress ramp and a descending stress ramp at equilibrium from 0 Pa up to a stress corresponding to a shear rate of 500 s⁻¹. These measurements make it possible to evaluate the viscosity of the studied systems for shear rates equal to 0.01 s⁻¹ and 100 s⁻¹.

Dynamic Measurements:

The limits of the linear viscoelastic domains were determined at 10⁻² and 1 Hz by subjecting the sample to a series of sinusoidal stresses of fixed frequency and of logarithmically distributed increasing amplitudes between two limits at a rate of 5 points per decade. The linear viscoelastic behaviour is then characterized by subjecting the sample to a series of 20 sinusoidal stresses of logarithmically distributed frequencies between 10 and 10⁻² Hz and of amplitudes such that the deformation amplitude is constant and located in the linear domain previously determined. These measurements make it possible to evaluate the complex modulus G* at 1 Hz and the loss angle δ at 1 Hz, in the linear viscoelastic domain. G* and δ are the viscoelastic parameters used to measure the physical properties of viscoelastic fluids, as explained in “An introduction to rheology” by H. A. Barnes, J. F. Hutton and K. Walters, pages 46 to 54 (published by Elsevier 1989). The modulus G* is equal to the square root of the sum of the squares of the elastic modulus G′ (storage modulus) and the loss modulus G″ (loss modulus). The tangent of the loss angle δ is equal to the ratio G″/G′.

1) Aqueous Gels:

The three gelling agents below were used in the dispersions given in the example:

Gelling Agent 1 (According to the Invention): Diblock copolymer of polystyrene 4 300 g/mol -b-sodium polyacrylate 25 460 g/mol (b=block). The glass transition temperature of the polystyrene block is equal to 76° C.

Gelling Agent 2 (Prior Art According to Document EP-A-1 279 398): diblock copolymer of polystyrene 1 800 g/mol -b-sodium polyacrylate 42 430 g/mol. The glass transition temperature of the polystyrene block is equal to 45° C.

Gelling Agent 3 (Prior Art): crosslinked sodium polyacrylate (Carbopol 980 sold by the company Noveon).

Preparation of the Gels: The aqueous gels comprising 2% (by weight) of gelling agents 1 and 2 are prepared by dissolving the appropriate amount of polymer in powder form in water, with stirring at 90° C. for 5 hours, in a hermetically sealed flask. The pH is adjusted to 7 by adding the appropriate amount of 2 mol/l sodium hydroxide. The gels obtained are transparent.

The aqueous gel comprising 2% (by weight) of gelling agent 3 is prepared by dissolving the appropriate amount of polymer in powder form in water, with stirring at 25° C. for 2 hours. The pH is adjusted to 7 by adding the appropriate amount of 2 mol/l sodium hydroxide. The gel obtained is transparent.

Rheological Characteristics of the Aqueous Gels

The three aqueous gels comprising 2% by weight of polymer were characterized according to the protocol indicated below. These gels have the following rheological characteristics:

| | Viscosity | Viscosity | | |
|---|---|---|---|---|
| | (0.01 s⁻¹) | (100 s⁻¹) | G* (1 Hz) | δ (1 Hz) |
| | (Pa · s) | (Pa · s) | (Pa) | (°) |
| Gelling agent 1 | 8 000 | 10 | 6 000 | 2 |
| Gelling agent 2 | 7 000 | 4 | 600 | 5 |
| Gelling agent 3 | 4 000 | 5 | 700 | 8 |

Gelling agent 1 has, at 2%, a water-gelling power greater than that of gelling agents 2 and 3, especially for low-amplitude stresses (high complex modulus and low loss angle).

2) Gelled Aqueous Dispersions of Nanotitanium Oxide at 5% by Weight

The three aqueous gels described above were used to obtain dispersions of nanotitanium oxide (TiO₂) comprising 5% by weight of TiO₂ relative to the total weight of the gel.

Preparation of the Dispersions: The TiO₂ particles are added to the aqueous gels described above, at 25° C., with stirring using an Ultraturrax homogenizer, for 5 minutes at a speed of 24 000 rpm. The spindle used has an outside diameter of 10 mm (reference 10F).

Stabilities of the TiO₂ Dispersions

The dispersions comprising 5% by weight of TiO₂ and 2% by weight of gelling agent relative to the total weight of the gel were subjected to an accelerated ageing test by centrifugation for 5 minutes at a speed of 28 000 rpm using a Beckman Optima TLX centrifuge equipped with a TLA 100.4 rotor. 0.5 gram of dispersion is taken from the top and the bottom of the centrifugation tubes; their solids content is then evaluated using a Sartorius MA 100 balance (measurement of the residual weight at 105° C.).

| | | Solids |
|---|---|---|
| | | content |
| | | (%) |
| Dispersion comprising gelling agent 1 | top of the tube | 7.0 |
| | bottom of the tube | 7.4 |
| Dispersion comprising gelling agent 2 | top of the tube | 3.2 |
| | bottom of the tube | 30.6 |
| Dispersion comprising gelling agent 3 | top of the tube | 5.9 |
| | bottom of the tube | 21.8 |

The results show that the dispersion of TiO₂ comprising 2% of gelling agent 1 used according to the invention remains stable after centrifugation and does not produce any sedimentation, in contrast with the dispersions comprising gelling agents 2 and 3 in which the mineral particles sedimented (differences in the proportions of solids content between the top and bottom of the tube). Thus, gelling agent 1 according to the invention gives an aqueous dispersion of TiO₂ that is more stable than those comprising gelling agents 2 and 3.

### Example 2

### According to the Invention and Comparative Example 3 Moisturizing Creams

| | Example 2 according | Comparative |
|---|---|---|
| Composition | to the invention | Example 3 |
| Oily phase: | | |
| Parleam oil | 20% | 20% |
| Aqueous phase: | | |
| Glycerol | 3% | 3% |
| Diblock copolymer of polystyrene | 1% | 0% |
| 4 300 g/mol -b-sodium | | |
| polyacrylate 25 460 g/mol | | |
| Crosslinked sodium polyacrylate | 0% | 1% |
| (Carbopol 980 sold by | | |
| the company Novéon) | | |
| Triethanolamine | 0 | 0.05% |
| Demineralized water | qs 100% | qs 100% |

Mode of preparation of the O/W emulsions: The polymers are dissolved in the aqueous phase by simple stirring for 5 hours at 90° C. for the block polymer and for 2 hours at 25° C. for the sodium polyacrylate. The oily phase is then gradually introduced into the aqueous phase at room temperature with stirring using an Ultra-turrax homogenizer, for 5 minutes at a speed of 24 000 rpm (reference 10F).

Characteristics of the emulsions: The emulsion of Example 2 according to the invention is a homogeneous white cream in which the mean size of the oil droplets is equal to 10 microns. The emulsion of Comparative Example 3 is macroscopically heterogeneous directly upon preparation, with formation of a surface oil film, corresponding to 30% of the oily phase, which, therefore, has not been emulsified.

Thus, the diblock polymer used according to the invention allows a surfactant-free emulsion comprising 20% oily phase to be stabilized, in contrast with the crosslinked sodium polyacrylate used in the prior art.

Example 4 According to the Invention: A cream can be prepared in a manner similar to that of Example 2, using the triblock polymer below instead of the diblock polymer: Polystyrene 4 300 g/mol/sodium polyacrylate 49 000 g/mol/polystyrene 4 300 g/mol. The weight proportion of the hydrophilic block is equal to 85%.

As used herein, where a certain polymer or polymer block is noted as being “obtained from” or “comprising”, etc. one or more monomers (or monomer units) this description is of the finished polymer or block material itself and the repeating units therein that make up, in whole or part, this finished product. One of ordinary skill in the art understands that, speaking precisely, a polymer or polymer block does not include individual, unpolymerized “monomers,” but instead is made up of repeating units derived from polymerized monomers.

The above written description of the invention provides a manner and process of making and using it such that any person skilled in this art is enabled to make and use the same, this enablement being provided in particular for the subject matter of the appended claims, which make up a part of the original description and including a composition for topical application, wherein it comprises at least one aqueous phase comprising at least one water-soluble or water-dispersible polymer chosen from (1) polymers of triblock structure B-A-B in which A is an ionic water-soluble polymer block and B is a hydrophobic polymer block, in which the amount of polymer block A is greater than or equal to 50% of the total weight of the triblock polymer B-A-B, and in which the hydrophobic block B has a glass transition temperature of greater than or equal to 70° C., and/or (2) polymers of diblock structure A-B, in which A is an ionic water-soluble polymer block and B is a hydrophobic polymer block, in which the amount of polymer block A is greater than or equal to 60% of the total weight of the diblock polymer A-B and in which the hydrophobic block B has a glass transition temperature of greater than or equal to 70° C. Preferred embodiments of the invention similarly fully described and enabled include the cosmetic use of a cosmetic composition according to the invention as a care product for the skin, the hair, the scalp, the eyelashes, the eyebrows, the nails or mucous membranes, as a makeup product, as an antisun product, as a rinse-out or leave-in hair product, or as a product for cleansing and/or removing makeup from the skin and/or the eyes.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition suitable for topical application comprising at least one aqueous phase comprising water and at least one water-soluble or water-dispersible polymer selected from the group consisting of:
(1) polymers of triblock structure B-A-B in which A is an ionic water-soluble polymer block and B is a hydrophobic polymer block, in which the amount of polymer block A is greater than or equal to 50% of the total weight of the triblock polymer B-A-B, and in which the hydrophobic block B has a glass transition temperature of greater than or equal to 70° C., and
(2) polymers of diblock structure A-B, in which A is an ionic water-soluble polymer block and B is a hydrophobic polymer block, in which the amount of polymer block A is greater than or equal to 60% of the total weight of the diblock polymer A-B and in which the hydrophobic block B has a glass transition temperature of greater than or equal to 70° C.

2. The composition according to claim 1, wherein the ionic water-soluble block A comprises one or more water-soluble monomers (Ia) or salts thereof, the monomers (Ia) being selected from the group consisting of:
(meth)acrylic acid,
styrenesulphonic acid,
vinylsulphonic acid,
(meth)allylsulphonic acid,
vinylphosphonic acid,
maleic acid,
itaconic acid,
crotonic acid,
dimethyldiallylammonium chloride,
methylvinylimidazolium chloride,
ethylenic carboxybetaines or sulphobetaines,
water-soluble vinyl monomers of formula (I): 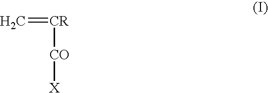
in which:
R is H, —CH₃, —C₂H₅ or —C₃H₇;
X is:
an alkyl oxide of the formula —OR₁ in which R₁ is a linear or branched, saturated or unsaturated hydrocarbon radical containing from 1 to 6 carbon atoms, substituted with at least one carboxylate, sulphonic, sulphate, phosphate and/or quaternary ammonium (—N⁺R₂R₃R₄) group, the radicals R₂, R₃ and R₄ being, independently of each other, a linear or branched, saturated or unsaturated hydrocarbon radical containing 1 to 6 carbon atoms, with the proviso that the sum of the carbon atoms of R₁+R₂+R₃+R₄ does not exceed 6; the radical R₁ optionally being substituted with a halogen atom; a hydroxyl; carboxylic acid; ether; primary amine; secondary amine (—NHR′); or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₁+R′+R″ does not exceed 6;
—NH₂,
—NHR₅, or
—NR₅R₆
wherein R₅ and R₆ are, independently of each other, a linear or branched, saturated or unsaturated hydrocarbon radical containing 1 to 6 carbon atoms, with the proviso that the total number of carbon atoms of R₅+R₆ does not exceed 6, the radicals R₅ and/or R₆ being substituted with at least one carboxylate, sulphonic, sulphate, phosphate and/or quaternary amine (—N⁺R₇R₈R₉) group, the radicals R₇, R₈ and R₉ being, independently of each other, a linear or branched, saturated or unsaturated hydrocarbon radical containing 1 to 6 carbon atoms, with the proviso that the sum of the carbon atoms of R₅+R₆+R₇+R₈+R₉ does not exceed 6; the radicals R₅ and/or R₆ optionally being substituted with a halogen atom; or a hydroxyl; ether; carboxylic acid; primary amine; secondary amine (—NHR′); or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₇+R₈+R′+R″ does not exceed 6.

3. The composition according to claim 1, wherein the ionic water-soluble block A comprises one or more monomers selected from the group consisting of neutral water-soluble monomers (Ib), hydrophobic monomers (Ic), and mixtures thereof.

4. The composition according to claim 3, wherein the ionic water-soluble block A comprises one or more neutral water-soluble monomers (Ib) selected from the group consisting of
(meth)acrylamide,
N-vinylacetamide,
N-methyl-N-vinylacetamide,
N-vinylformamide,
N-methyl-N-vinylformamide,
maleic anhydride,
vinylamine,
N-vinyllactams including a cyclic alkyl group containing from 4 to 9 carbon atoms,
vinyl alcohol,
water-soluble vinyl monomers of formula (II): 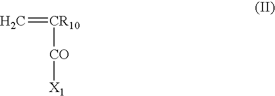
in which:
R₁₀ is H, —CH₃, —C₂H₅ or —C₃H₇;
X1 is:
an alkyl oxide of the formula —OR₁, in which R₁₁ is a linear or branched, saturated or unsaturated hydrocarbon radical containing from 1 to 6 carbon atoms, optionally substituted with a halogen atom; a hydroxyl (—OH); ether (—O—); primary amine (—NH₂); secondary amine (—NHR′) or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₁₁+R′+R″ does not exceed 6,
—NH₂,
—NHR₁₂, or p2 —NR₁₂R₁₃ in which R₁₂ and R₁₃ are, independently of each other, linear or branched, saturated or unsaturated hydrocarbon radicals containing 1 to 6 carbon atoms, with the proviso that the total number of carbon atoms of R₁₂+R₁₃ does not exceed 6, the radicals R₁₂ and R₁₃ optionally being substituted with a halogen atom; a hydroxyl (—OH); ether (—O—); primary amine (—NH₂); secondary amine (—NHR′); or tertiary amine (—NR′R″) group, the radicals R′ and R″ being, independently of each other, a linear or branched, saturated or unsaturated hydrocarbon radical containing 1 to 6 carbon atoms, with the proviso that the sum of the carbon atoms of R₁₂+R₁₃+R′+R″ does not exceed 6.

5. The composition according to claim 3, wherein the ionic water-soluble block A comprises one or more hydrophobic monomers (Ic) selected from the group consisting of:
styrene and its derivatives;
vinyl acetate;
vinyl ethers of formula CH₂═CHOR₁₄ in which R₁₄ is a linear or branched, saturated or unsaturated hydrocarbon radical containing from 1 to 6 carbon atoms;
acrylonitrile;
caprolactone;
vinyl chloride:
vinylidene chloride;
unsaturated silicone monomers;
hydrophobic vinyl monomers of formula (III) below: 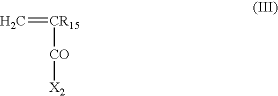
in which:
R₁₅ is H, —CH₃, —C₂H₅ or —C₃H₇;
X₂ is:
an alkyl oxide of the formula —OR₁₆ in which R₁₆ is a linear or branched, saturated or unsaturated hydrocarbon radical containing from 1 to 6 carbon atoms;
—NH₂,
—NHR₁₇, or
—NR₁₇R₁₈ in which R₁₇ and R₁₈ are, independently of each other, linear or branched, saturated or unsaturated hydrocarbon radicals containing 1 to 6 carbon atoms, with the proviso that the total number of carbon atoms of R₁₇+R₁₈ does not exceed 6.

6. The composition according to claim 1, wherein the ionic water-soluble block A is an ionic water-soluble polymer.

7. The composition according claim 6, wherein the ionic water-soluble block A is polyethyleneimine.

8. The composition according to claim 1, wherein the ionic water-soluble block A is totally or partially neutralized with a mineral or organic base.

9. The composition according to claim 1, wherein the hydrophobic block B comprises one or more hydrophobic monomers (Id) selected from the group consisting of:
styrene and its derivatives,
vinyl acetate,
vinyl ethers of formula CH₂═CHOR₂₅ in which R₂₅ is a linear or branched, saturated or unsaturated hydrocarbon radical containing from 1 to 22 carbon atoms,
acrylonitrile,
vinyl chloride
vinylidene chloride,
caprolactone,
alkenes,
silicone derivatives, leading, after polymerization, to silicone polymers,
hydrophobic vinyl monomers of formula (IV) below: 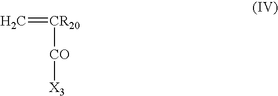
in which:
R₂₀ is H, —CH₃, —C₂H₅ or —C₃H₇;
X₃ is:
an alkyl oxide of the formula —OR₂₁ in which R₂₁ is a linear or branched, saturated or unsaturated hydrocarbon radical containing from 1 to 22 carbon atoms, optionally substituted with a halogen atom; a sulphonic (—SO₃⁻), sulphate (—SO₄⁻), phosphate (—PO₄H₂⁻), hydroxyl (—OH), ether (—O—), primary amine (—NH₂), secondary amine (—NHR₂₂), tertiary amine (—NR₂₂R₂₃) or quaternary ammonium (—N⁺R₂₂R₂₃R₂₄) group, the radicals R₂₂, R₂₃ and R₂₄ being, independently of each other, a linear or branched, saturated or unsaturated hydrocarbon radical containing 1 to 22 carbon atoms, with the proviso that the sum of the carbon atoms of R₂₁+R₂₂+R₂₃+R₂₄ does not exceed 22; it also being possible for R₂₁ to be a perfluoroalkyl radical, p2 —NH₂,
—NHR₂₅ or
—NR₂₅R₂₆ in which the radicals R₂₅ and R₂₆ are, independently of each other, a linear or branched, saturated or unsaturated hydrocarbon radical containing 1 to 22 carbon atoms, with the proviso that the total number of carbon atoms of R₂₅+R₂₆ does not exceed 22, the radicals R₂₅ and R₂₆ optionally being substituted with a halogen atom; a hydroxyl (—OH), ether (—O—), carboxylate (—CO₂⁻), sulphonic (—SO₃⁻), sulphate (—SO₄⁻), phosphate (—PO₄H₂⁻), carboxylic acid (—COOH), primary amine (—NH₂), secondary amine (—NHR′), tertiary amine (—NR′R″) or quaternary amine (—N+R′R″R′″) group, the radicals R′, R″ and R′″ representing, independently of each other, a linear or branched, saturated or unsaturated hydrocarbon radical containing 1 to 22 carbon atoms, with the proviso that the sum of the carbon atoms of R₂s +R₂₆+R′+R″+R′″ does not exceed 22; R₂₅ and R₂₆, independently of each other, may also be a perfluoroalkyl radical.

10. The composition according to claim 9, wherein the hydrophobic block B further comprises one or more ionic or neutral water-soluble monomers (Ie) selected from the group consisting of the following monomers, or salts thereof:
(meth)acrylic acid;
styrenesulphonic acid;
vinylsulphonic acid
(meth)allylsulphonic acid;
vinylphosphonic acid;
maleic anhydride;
maleic acid;
itaconic acid;
crotonic acid;
dimethyldiallylammonium chloride;
methylvinylimidazolium chloride;
(meth)acrylamide;
N-vinylacetamide
N-methyl-N-vinylacetamide;
N-vinylformamide
N-methyl-N-vinylformamide;
N-vinyllactams including a cyclic alkyl group containing from 4 to 9 carbon atoms;
vinyl alcohol;
2-vinylpyridine
4 -vinylpyridine;
water-soluble vinyl monomers of formula (V): 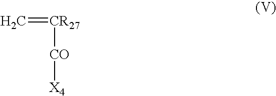
in which:
R₂₇ is H, —CH₃, —C₂H₅ or —C₃H₇;
X₄ is:
alkyl oxides of the formula —OR₂₈ in which R₂₈ is a linear or branched, saturated or unsaturated hydrocarbon radical containing from 1 to 6 carbon atoms, optionally substituted with a halogen atom; a sulphonic (—SO₃⁻), sulphate (—SO₄⁻), phosphate (—PO₄H₂⁻), hydroxyl (—OH), ether (—O—), primary amine (—NH₂), secondary amine (—NHR′), tertiary amine (—NR′R″) or a quaternary amine (—N+R′R″R′″) group, the radicals R″, R″ and R′″ being, independently of each other, a linear or branched, saturated or unsaturated hydrocarbon radical containing 1 to 6 carbon atoms, with the proviso that the sum of the carbon atoms of R₂₈+R′+R″+R′″ does not exceed 6,
—NH₂,
—NHR₂₉ or
—NR₂₉R₃₀ in which the radicals R₂₉ and R₃₀ are, independently of each other, linear or branched, saturated or unsaturated hydrocarbon radicals containing 1 to 6 carbon atoms, with the proviso that the total number of carbon atoms of R₂₉+R₃₀ does not exceed 6, the radicals R₂₉ and/or R₃₀ optionally being substituted with a halogen atom; a sulphonic (—SO₃⁻); sulphate (SO₄⁻); phosphate (—PO₄H₂⁻); hydroxyl (—OH); ether (—O—); primary amine (—NH₂); secondary amine (—NHR′); tertiary amine (—NR′R″) or a quaternary amine (—N+R′R″R′″) group, the radicals R′, R″ and R′″ representing, independently of each other, a linear or branched, saturated or unsaturated hydrocarbon radical containing 1 to 6 carbon atoms, with the proviso that the sum of the carbon atoms of R₂₉+R₃₀+R′+R″+R′″ does not exceed 6.

11. The composition according to claim 1, wherein the polymer has a molar weight of 1 000 g/mol to 500 000 g/mol.

12. The composition according to claim 1, comprising a diblock polymer, and wherein the proportion of the ionic hydrophilic block A in the diblock polymer is greater than 70% by weight relative to the total weight of the blocks A and B.

13. The composition according to claim 1, comprising a triblock polymer, and wherein the proportion of the ionic hydrophilic block A in the triblock polymer is greater than 60% by weight relative to the total weight of the blocks A and B.

14. The composition according to claim 1, wherein the polymer comprises sodium polyacrylate as block A and polystyrene as block B.

15. The composition according to claim 1, wherein the amount of polymer(s) ranges from 0.01% to 20% by weight relative to the total weight of the composition.

16. The composition according to claim 1, further comprising at least one oily phase.

17. The composition according to claim 16, further comprising solid particles selected from the group consisting of physical sunblock screening agents, pigments, fillers, and mixtures thereof.

18. The composition according to claim 1, wherein said composition constitutes a cosmetic or dermatological composition.

19. The composition according to claim 1, wherein said composition constitutes a care, treatment, protective, cleansing, makeup-removing and/or makeup product for keratin materials.

20. A method, comprising applying the composition of claim 1 to at least one of the skin, the hair, the scalp, the eyelashes, the eyebrows, the nails and mucous membranes, as a makeup product, as an antisun product, as a rinse-out or leave-in hair product, or as a product for cleansing and/or removing makeup.

21. A method for treating a keratin material, wherein the cosmetic composition as defined in claim 1 is applied to keratin material.

22. A method for stabilizing a cosmetic and/or dermatological composition comprising at least one aqueous phase and solid particles, said method comprising adding thereto at least one water-soluble or water-dispersible polymer selected from the group consisting of:
(1) polymers of triblock structure B-A-B in which A is an ionic water-soluble polymer block and B is a hydrophobic polymer block, in which the amount of polymer block A is greater than or equal to 50% of the total weight of the triblock polymer B-A-B, and in which the hydrophobic block B has a glass transition temperature of greater than or equal to 70° C., and
(2) polymers of diblock structure A-B, in which A is an ionic water-soluble polymer block and B is a hydrophobic polymer block, in which the amount of polymer block A is greater than or equal to 60% of the total weight of the diblock polymer A-B and in which the hydrophobic block B has a glass transition temperature of greater than or equal to 70° C.
